(19) United States
(12) Patent Application Publication
(10) Pub. No.: US 2007/0249055 A1
(43) Pub. Date: Oct. 25, 2007

(54) Method of measuring lotion and additive ingredient transfer

(75) Inventor: Miranda Aref Farage, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/409,865

(22) Filed: Apr. 24, 2006

(51) Int. Cl. G01N 33/44

(52) U.S. Cl. 436/85

(57) ABSTRACT

A method of determining the amount of lotion and/or additive ingredients transferred from a lotion and/or additive ingredient containing fibrous structure to the skin of a wearer is provided. The method simulates actual wear of a fibrous structure, such as a feminine hygiene pad to produce an accurate measurement of lotion and/or additive ingredient transfer to the skin of a wearer by determining an amount of lotion and/or additive ingredient transferred to a collecting device.

### FIELD OF THE INVENTION

The present invention relates to test methods for determining the amount of lotion and/or additive ingredients transferred from one surface to another surface. More particularly, the present invention relates to determining the amount of lotion and/or additive ingredients transferred from the surface of a lotion and/or additive ingredient containing fibrous structure to the surface of a collecting film.

### BACKGROUND OF THE INVENTION

Lotions of various types are known to provide various skin benefits, such as prevention or treatment of diaper rash. These lotions can be applied to the topsheet of fibrous structures, for example, and can be transferred to the body of the wearer during use. The addition of lotion to the topsheet of fibrous structures is known to provide benefits such as easier BM clean up on babies. Likewise, lotion on topsheets is known to provide for better skin health of babies, such as the reduction of diaper rash.

Worldwide, millions of women rely on disposable menstrual pads and pantiliners for feminine hygiene protection. Manufacturers of such products rely on comprehensive programs to ensure the safety of each material used in pads and pantiliners, and to confirm safety during use. Studies have included evaluations of dermatologic parameters to confirm that products have no adverse skin effects during use, and microbiological evaluations to ensure that the disposable menstrual pads and pantiliners do not alter vaginal or labial microflora. Clinical studies conducted on pads and pantiliners over the past 20 years have confirmed the overall safety of these products.

Despite rigorous testing programs demonstrating overall skin safety, many women experience sensations of discomfort or negative feelings while using pads during their period. These are typically described as feelings of rubbing or chafing, itching, tenderness, or the feeling of a rash. Such sensations are likely the result of the mechanical irritation associated with wearing any product in close proximity to the skin of the urogenital region for extended periods of time. Skin protectants and lotions on catamenial pads have been studied as a means of ameliorating some of these unpleasant sensations pad users experience. However, outside of clinical testing there are no means available for the measurement of lotion transfer between a fibrous structure and the skin of a wearer.

Accordingly there is a need for a means of quantifying protectant lotion transfer that can be used in place of costly clinical studies.

### SUMMARY OF THE INVENTION

A method for determining an amount of lotion and/or additive ingredient transferred from one surface to another surface is provided which comprises the steps of providing at least one lotion and/or additive ingredient containing fibrous structure, providing at least one collecting device, positioning the at least one collecting device on a surface of a wearer, contacting a surface of the at least one lotion and/or additive ingredient containing fibrous structure with a surface of the at least one collecting device, and determining the amount of lotion and/or additive ingredient transferred from the surface of the at least one lotion and/or additive ingredient containing fibrous structure to the surface of the at least one collecting device.

A method for determining an amount of lotion and/or additive ingredient transferred from one surface to another surface is provided which comprises the steps of providing at least one lotion and/or additive ingredient containing fibrous structure, providing at least one collecting film having an outer surface oriented towards the at least one lotion and/or additive ingredient containing fibrous structure and an inner surface oriented towards a surface of a wearer, positioning the inner surface of the at least one collecting film on the surface of the popliteal fossa of the wearer, contacting a surface of the at least one lotion and/or additive ingredient containing fibrous structure with the outer surface of the at least one collecting film, and determining the amount of lotion and/or additive ingredient transferred from the surface of the at least one lotion and/or additive ingredient containing fibrous structure to the outer surface of the at least one collecting film.

### BRIEF DESCRIPTION OF THE DRAWINGS

FIG. 1 shows the horizontal placement of two collecting devices along the fold at the back of the knee.

FIG. 2 is a graph illustrating the amount of lotion transferred from lotion and/or additive ingredient containing fibrous structures (feminine hygiene pads) containing differing amounts of lotion.

### DETAILED DESCRIPTION OF THE INVENTION

The present invention is directed to a method of determining the amount of lotion and/or additive ingredient transferred from a lotion and/or additive ingredient containing fibrous structure to the skin of a wearer. The method imitates wear of a lotion and/or additive ingredient containing fibrous structure, such as a feminine hygiene pad, for its intended use in the intended location to produce an accurate measurement of the amount of lotion and/or additive ingredient transferred to a collecting device used to simulate the skin of a wearer.

Collecting devices may be applied to the surface of a wearer on, above and/or below the fold at the back of the right or left knee. The surface located at the back of the knee is known as the popliteal fossa. The surface of the back of the knee may be the skin of the wearer or some intermediate material or layers of intermediate material between the collecting device and the skin of the wearer. As compared to other areas of the human body the popliteal fossa has the following characteristics, which make it a suitable test site for determining the amount of lotion and/or additive ingredient transferred from a lotion and/or additive ingredient containing fibrous structure to the skin of a wearer. Its location on the human body allows for prolonged contact with the skin, and normal movement from daily activity results in friction. As the wearers go through their everyday activities, normal movements generate friction between the lotion and/or additive ingredient containing fibrous structure and the collecting device at the surface of the back of the knee, thereby adding the element of mechanical irritation and imitating wear of a lotion and/or additive ingredient containing fibrous structure for its intended use in the intended location.

Lotion and/or additive ingredient containing fibrous structures may be placed over and in contact with the collecting devices, and held in place by any known means. When used behind the knee an elastic knee band of the appropriate size (for example Ace® Brand Knee Braces, Franklin Lakes, N.J., or Mueller Sport Care Elastic Knee Braces, Prairie du Sac, Wis.) may be used. The lotion and/or additive ingredient containing fibrous structures are removed after a set period of time, in certain embodiments the set period of time may be from about 0.5 hours to about 24 hours and in other embodiments from about 1 hour to about 20 hours after placement of the collecting devices and lotion and/or additive ingredient containing fibrous structure. In certain embodiments the collecting devices are then also removed from the skin of the wearer. In certain other embodiments the collecting devices are left on the surface of the wearer and a further lotion and/or additive ingredient containing fibrous structure is applied for an additional amount of time which in certain embodiments may be from about 0.5 hours to about 24 hours and in other embodiments from about 1 hour to about 20 hours after which both the collecting device and the lotion and/or additive ingredient containing fibrous structure may be removed or the collecting device may be left on the surface of the wearer and another lotion and/or additive ingredient containing fibrous structure applied.

When the collecting device is removed, it is then analyzed to determine the amount of lotion and/or additive ingredient transferred. It is to be understood that “determining the amount of lotion and/or additive ingredient transferred” encompasses both lotion transfer as a whole and the amount of individual components such as additive ingredients, which may be added to the lotion composition before the lotion is added to the fibrous structure, they may be added to the fibrous structure separately from the lotion, or only additive ingredients are added to the fibrous structure.

The collecting devices may be comprised of nonwoven fibrous webs, woven fibrous webs, knits or films (collecting films). In certain embodiments the collecting devices are comprised of translucent or transparent polymeric films. In certain other embodiments the collecting device transmits moisture vapor at a rate equal to or greater than human skin.

The collecting device may be conformable to anatomical surfaces. This means that when the collecting device is applied to a human anatomical surface it conforms to the surface even when the surface is moved. When a joint such as a knee is flexed and then returned to its unflexed position, the collecting device can flex to accommodate the flexion of the joint but can be resilient enough to continue to conform to the joint when the joint is returned to its unflexed condition.

In one embodiment the collecting films are from about 12 μm to about 25 μm thick. The collecting films may be comprised of but are not limited to at least one of polyurethane, polyester, polyethylene such as chlorinated polyethylene, styrene/butadiene block copolymers, polyether block amides, polyvinyl chloride or mixtures thereof.

In certain embodiments the collecting device may have an adhesive applied to at least one surface of the collecting device. The adhesive substantially secures the collecting device to the surface of a wearer, preventing the collecting device from moving or becoming detached from the surface of the wearer. The adhesive may be comprised of but not limited to at least one of acrylate copolymers such as iso-octyl acrylate:acrylamide copolymer, iso-octyl acrylate:n-vinyl pyrrolidone copolymer, crosslinked acrylates or mixtures thereof.

A fibrous structure is a physical object that comprises one or more fibers, natural and/or synthetic and upon which and/or within which lotion and/or one or more additive ingredients may be deposited. A lotion and/or additive ingredient containing fibrous structure is a fibrous structure containing lotion and/or one or more additive ingredients. Nonlimiting examples of fibrous structures include feminine care products (feminine hygiene pads, catamenial tampons, wipes), adult incontinence products, sanitary tissue products (facial tissue, toilet tissue, paper towels, wipes), baby care products (diapers, wipes), fabrics and home care products (cleaning wipes, dusting wipes), beauty care products such as wipes.

The lotion and/or additive ingredient containing fibrous structure may include at least one of embossed, pattern-densified, creped, uncreped, or combinations thereof. In another embodiment the lotion and/or additive ingredient containing fibrous structure may include at least one of a nonwoven web, cellulosic fiber containing web or combinations thereof. In one embodiment, the fibrous structure is a single- or multi-ply sanitary tissue product.

Lotion compositions may comprise emollients, immobilizing agents, and/or additive agents. The lotion compositions may be in the form of emulsions and/or dispersions. The lotion compositions may contain solids, gel structures, polymeric material, a multiplicity of phases (such as oily and water phase) and/or emulsified components. The lotion compositions may be shear thinning and/or they may strongly change their viscosity around skin temperature to allow for transfer and easy spreading on a user's skin. The lotion compositions may be semi-solid or of high viscosity so they do not substantially flow without activation during the life of the product or gel structures. The lotion composition may sooth, moisturize, and/or lubricate a user's skin.

Emollients as used herein, are materials that soften, soothe, supple, coat, lubricate, moisturize, or cleanse the skin and may include but are not limited to at least one of glycols (such as propylene glycol and/or glycerine), polyglycols (such as triethylene glycol), petroleum-based materials, fatty acids, fatty alcohols, fatty alcohol ethoxylates, fatty alcohol esters and fatty alcohol ethers, fatty acid ethoxylates, fatty acid amides and fatty acid esters, alkyl ethoxylates, hydrocarbon oils (such as mineral oil), squalane, fluorinated emollients, silicone oil (such as dimethicone) or mixtures thereof.

Petroleum-based emollients include 16 to 32 carbon atom hydrocarbons, or mixtures of 16 to 32 carbon atom hydrocarbons. Petroleum based hydrocarbons having these chain lengths include petrolatum (also known as “mineral wax,” “petroleum jelly” and “mineral jelly”). Petrolatum usually refers to more viscous mixtures of hydrocarbons having from 16 to 32 carbon atoms. A Petrolatum that may be used is available from Witco, Corp., Greenwich, Conn. as White Protopet® 1 S.

Fatty acid ester emollients that may be used include but are not limited to those derived from long chain C₁₂-C₂₈ fatty acids, such as C₁₆-C₂₂ saturated fatty acids, and short chain C₁-C₈ monohydric alcohols, such as C₁-C₃ monohydric alcohols. Nonlimiting examples of fatty acid ester emollients include but are not limited to at least one of methyl palmitate, methyl stearate, isopropyl laurate, isopropyl myristate, isopropyl palmitate, ethylhexyl palmitate, or mixtures thereof. Fatty acid ester emollients can also be derived from esters of longer chain fatty alcohols (C₁₂-C₂₈, such as C₁₂-C₁₆) and shorter chain fatty acids e.g., lactic acid, such as lauryl lactate and cetyl lactate.

Alkyl ethoxylate type emollients may include but are not limited to at least one of C₁₂-C₁₈ fatty alcohol ethoxylates having an average of from 3 to 30 oxyethylene units, such as from about 4 to about 23. Nonlimiting examples of such alkyl ethoxylates include laureth-3 (a lauryl ethoxylate having an average of 3 oxyethylene units), laureth-23 (a lauryl ethoxylate having an average of 23 oxyethylene units), ceteth-10 (acetyl ethoxylate having an average of 10 oxyethylene units), steareth-2 (a stearyl ethoxylate having an average of 2 oxyethylene units), steareth-10 (a stearyl ethoxylate having an average of 10 oxyethylene units) or mixtures thereof. These alkyl ethoxylate emollients may be used in combination with the petroleum-based emollients, such as petrolatum, at a weight ratio of alkyl ethoxylate emollient to petroleum-based emollient of from about 1:1 to about 1:3 in certain embodiments, from about 1:1.5 to about 1:2.5 in certain other embodiments.

Lotion compositions may include an “immobilizing agent,” which act to prevent migration of the emollient so that it can remain primarily on the surface of the fibrous structure to which it is applied so that it may deliver maximum softening benefit as well as be available for transferability to the user's skin.

Immobilizing agents may include agents that prevent migration of the emollient into the fibrous structure such that the emollient remains primarily on the surface of the fibrous structure therefore facilitating transfer of the lotion composition to a user's skin. Immobilizing agents may function as viscosity increasing agents and/or gelling agents.

Immobilizing agents may include but are not limited to at least one of waxes such as ceresin wax, ozokerite, microcrystalline wax, petroleum waxes, fisher tropsh waxes, silicone waxes, paraffin waxes, polyethylene waxes, beeswax, fatty alcohols such as cetyl, cetaryl, cetearyl and/or stearyl alcohol, fatty acids and their salts such as metal salts of stearic acid, mono and polyhydroxy fatty acid esters, mono and polyhydroxy fatty acid amides, silica and silica derivatives, gelling agents, thickeners or mixtures thereof.

An additive ingredient may be added to the lotion composition before the lotion is added to the fibrous structure, it may be added to the fibrous structure separately from the lotion, or only additive ingredient is added to the fibrous structure. In certain embodiments the additive ingredient either in combination with the lotion composition or separately from the lotion composition may be added into the fibrous structure or on to the surface of the fibrous structure.

Nonlimiting examples of additive ingredients that may be incorporated on and/or in the fibrous structure comprise at least one of surface treating composition, nanotechnology agents, encapsulated time release agents, skin healants, perfumes, such as long lasting and/or enduring perfumes, antibacterial agents, antiviral agents, botanical agents, disinfectants, pharmaceutical agents, film formers, dyes, colorants, surfactants, absorbents, permanent wet strength agents, temporary wet strength agents, deodorants, opacifiers, astringents, solvents, cooling sensate agents, such as camphor, thymol, menthol or mixtures thereof.

The surface treating composition may increase or decrease the surface friction of the surface of the fibrous structure. In certain embodiments, the surface treating composition will reduce the surface friction of the surface of the fibrous structure compared to a surface of the fibrous structure without such surface treating composition.

The surface treating composition may be a composition comprised of one or more surface treating agents that improves the tactile sensation of a surface of a fibrous structure as perceived by a user whom holds the fibrous structure and rubs it across the user's skin. Such tactile perceivable softness can be characterized by, but is not limited to, friction, flexibility, and smoothness, as well as subjective descriptors, such as a feeling like lubricious, velvet, silk or flannel. The surface treating composition may or may not be transferable. In certain embodiments, it may be substantially non-transferable.

Examples of surface treating agents may include but are not limited to at least one of polymers such as polyethylene and derivatives thereof, hydrocarbons, oils, silicones, siloxanes, organosilicones, quaternary ammonium compounds, ester-functional quaternary ammonium compounds, fluorocarbons, substituted C₁₀-C₂₂ alkanes, substituted C₁₀-C₂₂ alkenes, in certain embodiments the substituted C₁₀-C₂₂ alkenes may be derivatives of fatty alcohols, polyols, derivatives of polyols such as esters and ethers, sugar derivatives such as ethers and esters or mixtures thereof.

Oils include but are not limited to at least one of mineral oil, silicone oil, silicone gels or mixtures thereof. Silicones include but are not limited to at least one of polydimethylsiloxanes, aminosilicones, cationic silicones, quaternary silicones, silicone betaines or mixtures thereof. In certain embodiments the siloxane may be an aminofunctional polydimethylsiloxane such as aminoethylaminopropyl polydimethylsiloxane.

Quaternary ammonium compounds that may be used in the present invention include but are not limited to at least one of dialkyldimethylammonium salts such as ditallowdimethylammonium chloride, ditallowdimethylammonium methylsulfate, di(hydrogenated tallow)dimethylammonium chloride or mixtures thereof. In one example, the surface treating composition comprises di(hydrogenated tallow)dimethylammonium chloride, commercially available from Witco Chemical Company Inc. of Dublin, Ohio as Varisoft 137®.

Ester-functional quaternary ammonium compounds that may be used in the present invention include but are not limited to at least one of diester dialkyl dimethyl ammonium salts such as diester ditallow dimethyl ammonium chloride, monoester ditallow dimethyl ammonium chloride, diester ditallow dimethyl ammonium methyl sulfate, diester di(hydrogenated)tallow dimethyl ammonium methyl sulfate, diester di(hydrogenated)tallow dimethyl ammonium chloride, or mixtures thereof. In one embodiment, the surface treating composition comprises diester ditallow dimethyl ammonium chloride and/or diester di(hydrogenated)tallow dimethyl ammonium chloride, both commercially available from Witco Chemical Company Inc. of Dublin, Ohio under the tradename “ADOGEN SDMC™”.

In one embodiment, the surface treating composition of the present invention comprises a microemulsion and/or a macroemulsion of a surface treating agent in water. In such an example, the concentration of the surface treating agent within the surface treating composition may be from about 3% to about 60% and/or from about 4% to about 50% and/or from about 5% to about 40%. Nonlimiting examples of such microemulsions are commercially available from Wacker Chemie AG München, Germany (MR1003, MR103, MR102). A nonlimiting example of such a macroemulsion is commercially available from General Electric Silicones, Wilton, Conn. (CM849).

Nanotechnology agents are organic or inorganic particles having average diameters of about 500 μm or less. In one example, particle size distributions of the nanotechnology agents in the present invention may fall anywhere within the range from about 2 μm to less than about 500 μm, alternatively from about 2 μm to less than about 100 μm, and alternatively from about 2 μm to less than about 50 μm. In certain embodiments nanotechnology agents can also include crystalline or amorphous materials.

Inorganic nanotechnology agents may include but are not limited to at least one of oxides such as inorganic metal oxides, silicates such as layered clay minerals, carbonates and hydroxides

Inorganic metal oxides may be natural or synthetic and generally fall within two groups-photoactive and non-photoactive nanotechnology agents. General examples of photoactive metal oxide nanotechnology agents include zinc oxide and titanium oxide. Photoactive metal oxide nanotechnology agents require photoactivation from either visible light (e.g. zinc oxide) or from UV light (e.g. titanium oxide). Zinc oxide coatings have generally been used as anti-microbial agents or as anti-fouling agents.

Non-photoactive metal oxide nanotechnology agents do not use UV or visible light to produce the desired effects. Examples of non-photoactive metal oxide nanotechnology agents include, but are not limited to silica and alumina nanotechnology agents, and mixed metal oxide nanotechnology agents including, but not limited to saponites, and hydrotalcite. Aluminum can be found in many naturally occurring sources, such as kaolinite and bauxite.

Layered clay minerals may be either naturally occurring or synthetic and include those in the geological classes of smectites, kaolins, illites, chlorites, attapulgites and mixed layer clays. Variants and isomorphic substitutions of these layered clay minerals offer unique applications. Smectites, include montmorillonite, bentonite, pyrophyllite, hectorite, saponite, sauconite, nontronite, talc, beidellite, volchonskoite and vermiculite. Kaolins include kaolinite, dickite, nacrite, antigorite, anauxite, halloysite, indellite and chrysotile. Illites include bravaisite, muscovite, paragonite, phlogopite and biotite. Chlorites include corrensite, penninite, donbassite, sudoite, pennine and clinochlore. Attapulgites include sepiolite and polygorskyte. Mixed layer clays include allevardite and vermiculitebiotite.

One or more skin healants may be included. Skin healants include but are not limited to at least one of zinc oxide, vitamins, such as Vitamin B3, Vitamin E, sucrose esters of fatty acids, anti-inflammatory compounds, lipids, inorganic anions, inorganic cations, protease inhibitors, sequestration agents, chamomile extracts, aloe vera, calendula officinalis, alpha bisalbolol, or mixtures thereof.

Testing is conducted using lotion and/or additive ingredient containing fibrous structures (feminine hygiene pads) to determine the amount of lotion transferred to a collecting device as a function of the amount of lotion on the feminine hygiene pads and total wear time.

To measure the amount of lotion transferred from lotion and/or additive ingredient containing fibrous structures to collecting devices, wearers are selected. Wearers are excluded if: 1) they have sunburn, acne, scar tissue or any other skin abnormality at the test sites, 2) they have active dermatitis, 3) they are taking anti-inflammatory, corticosteroids or other medications that may interfere with test results, 4) they experience irritation or discomfort in the area behind the knee, or 5) have diabetes, kidney, heart or circulatory disease, or leg varicosities. In addition, female subjects are excluded if they were pregnant.

Wearers are instructed to shave the area behind the knees prior to the test's start to allow the collecting device to adhere to the skin. Further, wearers are asked to refrain from using lotions, creams, or any other skin preparation in the test area, and to refrain from swimming and tanning/sun exposure for the duration of the test.

Collecting films such as Tegaderm™HP from 3M™, St. Paul, Minn. can be used as collecting devices for the transferred lotion. The films measured 6×3.5 cm.

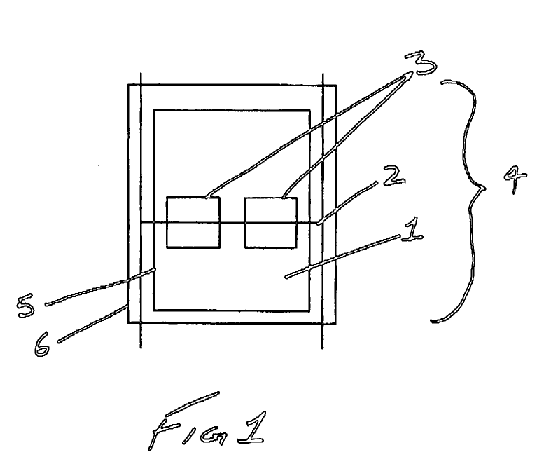

As shown in FIG. 1 two or more collecting devices 3 can be applied to the skin surface (popliteal fossa 1) of a wearer behind the knee 4. The collecting devices can be placed on, above and/or below the fold 2 at the back of the right or left knee 4, either one on top of another in a vertical arrangement or side by side in a horizontal arrangement, on the popliteal fossa 1 of one or both knees 4. A lotion and/or additive ingredient containing fibrous structure 5 can then be placed over the collecting devices 3 on the popliteal fossa 1 of the knee 4, and held in place by an elastic knee band of the appropriate size 6 (either Ace® Brand Knee Braces, Franklin Lakes, N.J., or Mueller Sport Care Elastic Knee Braces, Prairie du Sac, Wis.). The lotion and/or additive ingredient containing fibrous structure or structures 5 may be removed after about three hours after placement (to simulate the average amount of time a feminine hygiene pad is worn). Additionally, one or more of the collecting devices 3 may be removed after about three hours after placement. Fresh lotion and/or additive ingredient containing fibrous structures 5 can be applied over the remaining collecting device(s) 3 for about an additional three hours. Thus, a possible four or more collecting devices 3 (two or more from each knee) could be collected from each wearer, e.g., one or more collecting devices after about 3 hours and about 6 hours of exposure for both the left and right knee 4. The placement of the collecting devices 3 may be randomized as to vertical or horizontal arrangements and the right or left knee 4. Additionally, the removal of the collecting devices 3 at about three hours or about six hours may be randomized.

The lotion used in the test contains behenyl alcohol. Therefore to measure the amount of lotion transferred, the amount of behenyl alcohol transferred to the collecting device 3 is determined. Each collecting 3 device is removed with clean forceps and stored refrigerated in individual vials until analyzed. 50 mg (milligrams) of tricosanol (Nu-Chek Prep Inc., Elysian, Minn.) is dissolved in 1 L (liter) of Hexane (CHROMASOLV® Aldrich Inc., St. Louis, Mo.). 10 ml (milliliters) of the tricosanol/hexane solution is added to the collecting devices and mixed with gentle agitation for about 30 minutes. An aliquot of liquid from the mixture is filtered using disposable syringes with 13 mm (millimeter) GHP Acrodisc 0.45 μm (micrometer) filters (PerkinElmer Life and Analytical Sciences, Inc., Wellesley, Mass.). Aliquots (1 μl) are injected (325° C.) into an HP 5890 gas chromatograph (Hewlett-Packard Company, Palo Alto, Calif.) equipped with a Chrompack CP-Sil 5 CB column (0.25 mm ID, 0.25 μm film, 2m), (Varian, Inc., Palo Alto, Calif.) and a flame ionization detector. The peak area of the behenyl alcohol peak is used to calculate the amount of formulation transferred to the collecting devices.

A test was prepared to simulate the affect, if any that the lotion concentration on a lotion and/or additive ingredient containing fibrous structure which was a topsheet of a feminine hygiene pad might have on the transfer of the lotion to the skin of a wearer.

The lotion comprised:

| | % amt based on total |
|---|---|
| Components | weight of comp |
| Petrolatum | 75-85 |
| Behenyl Alcohol | 5-15 |
| Fumed Silica | 2-5 |
| Phytoconcentrol Chamomile Extract | 0.5-1 |
| Zinc Oxide | 5-10 |

amt = amount
comp = composition

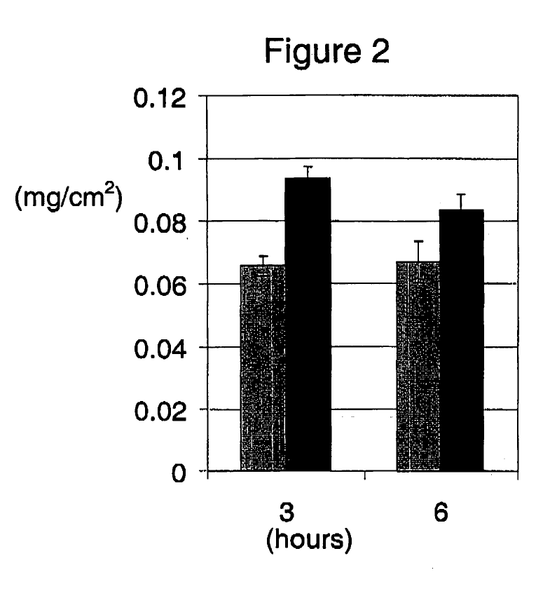

The topsheet of the feminine hygiene pad contained either 0.7 mg/cm² or 1.4 mg/cm² of the lotion. The topsheet comprised a hydrophobic spunbond bicomponent nonwoven. The bicomponent fibers are in a sheath form of a blend of polypropylene and polyethylene, with polyethylene on the outside. The collecting devices used were collecting films (Tegaderm™HP). A comparison of the amount of lotion transferred to the collecting films is shown in FIG. 2. After 3 hours of exposure, lotion transfer was significantly greater from the pad containing 1.4 mg/cm² of the lotion (sample 2) compared to the pad containing 0.7 mg/cm² of the lotion (sample 1). The application of a second set of pads for an additional 3 hours (i.e., 6 hours total) gave a similar result with the lotion transfer being significantly greater from the pad containing 1.4 mg/cm² of the lotion (sample 4) compared to the pad containing 0.7 mg/cm² of the lotion (sample 3).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

It will be understood that the embodiment(s) described herein is/are merely exemplary, and that one skilled in the art may make variations and modifications without departing from the spirit and scope of the invention. All such variations and modifications are intended to be included within the scope of the invention as described hereinabove. Further, all embodiments disclosed are not necessarily in the alternative, as various embodiments of the invention may be combined to provide the desired result.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for determining an amount of lotion and/or additive ingredient transferred from one surface to another surface comprising the steps of:
a. providing at least one lotion and/ or additive ingredient containing fibrous structure;
b. providing at least one collecting device;
c. positioning the at least one collecting device on a surface of a wearer;
d. contacting a surface of the at least one lotion and/or additive ingredient containing fibrous structure with a surface of the at least one collecting device; and
e. determining the amount of lotion and/or additive ingredient transferred from the surface of the at least one lotion and/or additive ingredient containing fibrous structure to the surface of the at least one collecting device.

2. The method of claim 1 wherein the lotion and/or additive ingredient containing fibrous structure comprises at least one of tampons, menstrual pads, interlabial pads, pantiliners, topsheets, diapers, training pants, adult incontinence products, sanitary tissues, facial tissues or wipes.

3. The method of claim 1 wherein the collecting device is at least one of nonwoven fibrous webs, woven fibrous webs, knits or films.

4. The method of claim 3 wherein the film is at least one of polyurethane, polyester, polyethylene, styrene/butadiene block copolymers, polyether block amides, polyvinyl chloride or mixtures thereof.

5. The method of claim 1 wherein the collecting device is positioned on the surface of the popliteal fossa of the wearer through the use of an adhesive.

6. The method of claim 5 wherein the adhesive comprises at least one of acrylate copolymers, crosslinked acrylates or mixtures thereof.

7. The method of claim 1 wherein the contacting is done for an amount of time within the range of about 0.5 hours to about 24 hours.

8. The method of claim 1 wherein the contacting is done for an amount of time within the range of about 1 hour to about 20 hours.

9. The method of claim 1 wherein the lotion is hydrophobic.

10. The method of claim 1 wherein the lotion is hydrophilic.

11. The method of claim 1 wherein the lotion and/or additive ingredient comprises at least one of emollients, immobilizing agents, surface treating compositions, nanotechnology agents, encapsulated time release agents, skin healants, perfumes, antibacterial agents, antiviral agents, botanical agents, disinfectants, pharmaceutical agents, film formers, dyes, colorants, surfactants, absorbents, permanent wet strength agents, temporary wet strength agents, deodorants, opacifiers, astringents, solvents, cooling sensate agents or mixtures thereof.

12. A method for determining an amount of lotion and/or additive ingredient transferred from one surface to another surface comprising the steps of:
a. providing at least one lotion and/or additive ingredient containing fibrous structure;
b. providing at least one collecting film having an outer surface oriented towards the at least one lotion and/or additive ingredient containing fibrous structure and an inner surface oriented towards a surface of a wearer;
c. positioning the inner surface of the at least one collecting film on the surface of the popliteal fossa of the wearer;
d. contacting a surface of the at least one lotion and/or additive ingredient containing fibrous structure with the outer surface of the at least one collecting film; and
e. determining the amount of lotion and/or additive ingredient transferred from the surface of the at least one lotion and/or additive ingredient containing fibrous structure to the outer surface of the at least one collecting film.

13. The method of claim 12 wherein the lotion and/ or additive ingredient containing fibrous structure is at least one of tampons, menstrual pads, interlabial pads, pantiliners, topsheets, diapers, training pants, adult incontinence products, sanitary tissues, facial tissues or wipes.

14. The method of claim 12 wherein the contacting is done for an amount of time within the range of about 0.5 hours to about 24 hours.

15. The method of claim 12 wherein the contacting is done for an amount of time within the range of about 1 hour to about 20 hours.

16. The method of claim 12 wherein the lotion and/or additive ingredient comprises at least one of emollients, immobilizing agents, surface treating compositions, nanotechnology agents, encapsulated time release agents, skin healants, perfumes, antibacterial agents, antiviral agents, botanical agents, disinfectants, pharmaceutical agents, film formers, dyes, colorants, surfactants, absorbents, permanent wet strength agents, temporary wet strength agents, deodorants, opacifiers, astringents, solvents, cooling sensate agents or mixtures thereof.

17. The method of claim 12 wherein the collecting film is at least one of polyurethane, polyester, polyethylene, styrenelbutadiene block copolymers, polyether block amides, polyvinyl chloride or mixtures thereof.

18. The method of claim 12 wherein the collecting film comprises an adhesive.

19. The method of claim 18 wherein the adhesive comprises at least one of acrylate copolymers, crosslinked acrylates or mixtures thereof.

20. The method of claim 12 wherein the collecting film is positioned on the surface of the popliteal fossa of the wearer through the use of an adhesive.
